(19) United States
(12) Patent Application Publication
(10) Pub. No.: US 2026/0241092 A1
(43) Pub. Date: Aug. 20, 2026

(54) ABSORBER MODULE TO PREVENT DEVICE CONTAMINATION

(71) Applicants: CUBIST CO., LTD., Seoul (KR); GENEONE LIFE SCIENCE, INC., Seoul (KR)

(72) Inventors: Young Woo OH, Seoul (KR); Young Kun PARK, Blue Bell, PA (US); Joel Maslow, Blue Bell, PA (US); Byung Mun CHO, Incheon (KR); Moon Sup JEONG, Incheon (KR); Hyo Jin LEE, Seongnam-si, Gyeonggi-do (KR); Gee Ho PARK, Seoul (KR); Yee Un OH, Goyang-si, Gyeonggi-do (KR)

(73) Assignees: CUBIST CO., LTD., Seoul (KR); GENEONE LIFE SCIENCE, INC., Seoul (KR)

(21) Appl. No.: 19/118,769

(22) PCT Filed: Nov. 11, 2022
(86) PCT No.: PCT/KR2022/017717

§ 371 Date: Apr. 6, 2025

(51) Int. Cl. A61M 1/00; A61M 39/20

(52) U.S. Cl. CPC A61M 1/78; A61M 1/88; A61M 39/20; A61M 2205/125

(57) ABSTRACT

The present disclosure relates to an absorber module that prevents contamination due to foreign substances and secretions, and more specifically, to an absorber module that prevents contamination due to foreign substances and secretions, the absorber module including: a housing that has a suction module therein and has an adjustment part being able to adjust the suction intensity on the outer surface; a suction part that is connected to the upper end of a body part, can generate negative pressure outside, and has a top surface having a flat shape; an annular first packing; and an annular second packing that is disposed at the upper end of the first packing and being attachable to and detachable from the first packing.

### TECHNICAL FIELD

The present disclosure relates to an absorber module that prevents device contamination due to foreign substances and secretions, and more specifically, to an absorber module that prevents device contamination due to foreign substances and secretions, the absorber module including: a housing that has a suction module therein and has an adjustment part being able to adjust the suction intensity on the outer surface; a suction part that is connected to the upper end of a body part, can generate negative pressure outside, and has a top surface having a flat shape; an annular first packing; and an annular second packing that is disposed at the upper end of the first packing and being attachable to and detachable from the first packing.

### BACKGROUND ART

The description of this section only provides the background information of the present disclosure without configuring the related art.

As shown in FIG. 1, a suction module 10 of the related art discloses a structure including a handheld handpiece, a tip mounted at the front end of the handpiece and configured to come into contact with the skin, and a dome-shaped protrusion 11 extending upward from the inside of the tip.

However, the suction module 10 of the related art has been pointed out to have a problem in that cleansing liquid, dead skin cells, or foreign substances are drawn into the protrusion 11 due to negative pressure, thereby frequently clogging a suction hole or a suction port.

In order to prevent this problem, a sponge or the like is embedded inside the protrusion 11 to block the inflow of foreign substances. However, since the protrusion is fixed to the suction module in a non-detachable manner, the embedded sponge cannot be replaced, and accordingly, foreign substances accumulate in the sponge and cause hygiene and cleanliness issues such as bacterial growth and odor generation.

In order to solve the problems of the suction module 10 of the related art described above, there have been cases where some domestic and overseas companies have conducted research on suction modules that restrict the shape of the cap, but the commercialization of such products has not been realized because the cost required to implement the cap-restricting structure is excessively high, or even if not, the effectiveness of the relevant device is not significant compared to its manufacturing cost. As a result, the market competitiveness of such modules is lower than that of the suction module 10 of the related art, and thus there have been no cases of full-scale commercialization.

Accordingly, as described above, there is a demand for the development of a suction module that prevents device contamination caused by secretions and can address the problems of the related art.

### DISCLOSURE OF INVENTION

### Technical Problem

The problems to be solved by the present disclosure is to complement the defects of the related art described above, and the objects of the present disclosure are as follows.

First, the present disclosure aims to provide an absorber module that prevents device contamination due to foreign substances and secretions, the absorber module preventing the ingress of foreign substances and secretions into the device by means of a filter part formed inside a cap disposed on the exterior of the device, and allowing for continuous cleanliness management by enabling the filter part to be removed and cleaned.

Second, the present disclosure aims to provide an absorber module that prevents device contamination due to foreign substances and secretions, the absorber module improving operability through a button part having a shape corresponding to the shape of the user's finger, and facilitating suction intensity adjustment through a plurality of panel parts supporting the button part.

The objects of the present disclosure are not limited to the objects described above and other objects will be clearly understood by those skilled in the art from the following description.

### Technical Solution

According to the present disclosure, there is provided an absorber module that prevents device contamination due to foreign substances and secretions, the absorber module including: a housing having a suction module therein and including an adjustment part, which can adjust suction intensity, on an outer surface thereof; a suction part connected to an upper end of a body part, being able to generate negative pressure outside, and having a flat top surface; and a cap blocking ingress of external substances by covering an outer surface of the suction part to be able to prevent the suction part from being contaminated by external substances, and being attachable to and detachable from an upper end of the suction part.

In this case, the cap may include: a body part detachably coupled to a side of the suction part; and a head part extending from a top surface of the body part and spaced apart from the upper end of the suction part by a predetermined distance when fastened to the suction part.

Further, the head part may include: an annular first packing extending from the upper end of the body part; an annular second packing disposed at an upper end of the first packing and being attachable to and detachable from the first packing; and a filter part disposed between the first packing and the second packing.

Further, the second packing may include a coupling projection protruding from a bottom surface of the second packing so that as a contact surface area between the first packing and the second packing primarily increases, a fastening force of the first packing and the second packing can be improved.

Meanwhile, the second packing may include an elastic part disposed between the second packing and the coupling projection to be able to prevent damage to the second packing due to impact that is generated during attachment and detachment.

In this case, the elastic part may include a protrusion protruding from a bottom surface of the elastic part so that as the contact surface area between the first packing and the second packing secondarily increases, the fastening force of the first packing and the second packing can be improved, and the first packing may include an accommodation groove disposed on a top surface thereof to be able to accommodate the protrusion.

Further, the protrusion may have a predetermined slope so that as the contact surface area between the first packing and the second packing tertiarily increases, the fastening force of the first packing and the second packing can be improved, and the accommodation groove may have an inclination corresponding to the slope of the protrusion so that the protrusion can be inserted therein.

According to another aspect of the present disclosure, the adjustment part may include a button part that allows a user to adjust the suction intensity of the suction part stepwise and selectively through pressing operations, and a panel part that is disposed on the bottom surface of the button part to be able to support the button part.

In this case, the button part, for the user's operational convenience, may include a first eccentric portion formed so that the height of the button part gradually increases toward its outer surface from the center of a button, and a second eccentric portion formed such that the height of the button part gradually decreases toward its outer surface from the outer surface of the first eccentric portion to guide the user to press the first eccentric portion.

Further, the second eccentric portion may have a shape in which its width gradually narrows downward from the top surface of the button part along the longitudinal axis of the button part to be able to prevent damage to the button part and the housing due to friction when the button part is pressed by a user.

Further, the panel part may include a first panel that is disposed on the bottom surface of the button part to be able to support the button part and primarily prevent external substances from entering the housing, a second panel that is disposed on the bottom surface of the first panel to be able to secondarily prevent external substances from entering the housing, and a third panel that is disposed on the bottom surface of the second panel to be able to support the first panel and the second panel.

Meanwhile, the first panel may include a first transfer portion that protrudes from the bottom surface of the first panel, thereby primarily transferring pressure applied downward to the button by a user to an adjustment module, a second transfer portion that is engraved on the bottom surface of the second panel, thereby secondarily transferring pressure applied downward to the first transfer portion to the adjustment module.

In this case, the first panel may include a guider that protrudes from the side of the first panel so that the first panel and the second panel can be fastened in a predetermined coupling direction, and the second panel may include a guider accommodation portion that protrudes from the side of the second panel to be able to accommodate the guider.

Further, the third panel may include a plurality of coupling grooves that protrude from the side of the third panel to be fixedly coupled to the adjustment module and include an accommodation space in which a coupling member, etc. can be inserted.

Additional configurations of the present disclosure will be partially described below and may be partially and easily known from the description or may be understood by embodiments of the present disclosure.

The above general description and the following detailed description are both provided only as examples without limiting the present disclosure described in the claims.

### Advantageous Effects

The effects of the present disclosure having the configuration described above are as follows.

First, by separating the cap, which is contaminated by skin debris after medical procedures and skincare, into the first packing and the second packing, each unit of the cap and the filter part can be cleaned, which enables continuous hygiene management.

Second, the operation feel is improved by the button part, which has a shape corresponding to the shape of the user's finger, and the adjustment of the suction intensity is facilitated by the plurality of panel parts supporting the button part.

Third, as the upper surface of the suction part is formed in a flat shape, it is easy to disinfect and maintain the suction part contaminated due to prolonged or continuous use.

The effects of the present disclosure are not limited to those described above and other effects not stated herein may be made apparent to those skilled in the art from the descriptions of the claim.

### BRIEF DESCRIPTION OF THE DRAWINGS

FIG. 1 is a perspective view of a suction module of the related art.

FIG. 2 is a perspective view of an absorber module that prevents device contamination due to foreign substances and secretions according to an embodiment of the present disclosure.

FIG. 3 is a detailed view of a cap according to an embodiment of the present disclosure.

FIG. 4 is a perspective view of an adjustment part according to an embodiment of the present disclosure.

FIG. 5 is a detailed view of a button part according to an embodiment of the present disclosure.

FIG. 6 is an exploded perspective view of a panel part according to an embodiment of the present disclosure.

FIG. 7 and FIG. 8 are detailed views of a suction part according to an embodiment of the present disclosure.

### DETAILED DESCRIPTION

Hereinafter, detailed embodiments of the present disclosure are described in detail with reference to the accompanying drawings.

However, in the following description of a detailed embodiment of the present disclosure, the detailed description of well-known functions or configurations relating to the present disclosure is omitted when it is determined to unnecessarily obscure the gist of the present invention.

The above described objects, features, and advantages of the present disclosure will be made clearer through the following detailed description related to the accompanying drawings. However, the present disclosure may be modified in various ways and implemented by various exemplary embodiments, so specific embodiments are illustrated in the drawings and described in detail hereafter.

Detailed descriptions of well-known functions or configurations relating to the present disclosure will not be provided so as not to unnecessarily obscure the gist of the present disclosure. Further, the numbers used in the description of this specification are merely identifiers for discriminating one component from another component.

In addition, the suffix “part” used for components in the following description is used only to facilitate the description of the specification, and does not have discriminate meanings or functions in itself.

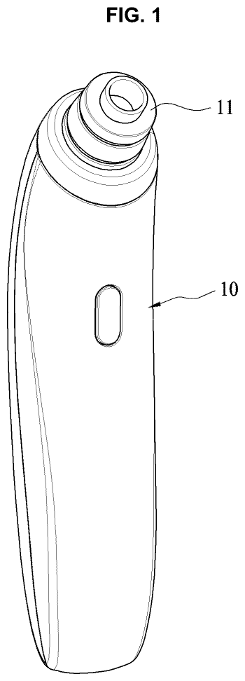

FIG. 1 is a perspective view of an absorber module that prevents device contamination due to foreign substances and secretions according to an embodiment of the present disclosure.

An absorber module that prevents device contamination due to foreign substances and secretions according to an embodiment of the present disclosure may include a housing 200, a suction part 300, and a cap 400.

The housing 200 may have a suction module therein.

The housing 200 may include an adjustment part 210 that can adjust suction intensity.

The housing 200 may be formed as a rigid body that does not change in shape (for example, a metal such as iron, aluminum, or copper, or a synthetic resin such as polystyrene (PS), ABS, polyacetal (POM), polyethylene (PE), polyvinyl chloride (PVC), polycarbonate (PC), polycaprolactone (PCL), and polypropylene (PP) may be selected).

The suction part 300 is connected to the upper end of the body part 410, can suck impurities such as sebum and blackheads from the skin surface, and can generate negative pressure externally.

The cap 400 covers the outer surface of the suction part 300 to prevent the suction part 300 from being contaminated by external substances, so it can block the ingress of external substances into the suction part 300.

The cap 400 can be attached to the upper end of the suction part 300 and can be detached from the upper end of the suction part 300.

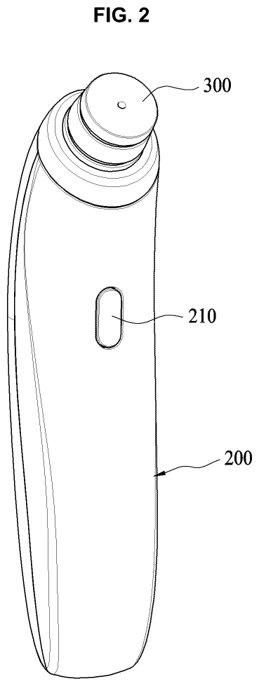

FIG. 2 is a detailed view of the cap 400 according to an embodiment of the present disclosure.

The cap 400 may include a body part 410 and a head part 420.

The body part 410 is detachably coupled to the side of the suction part 300 and the outer surface may be knurled for improved grip.

The head part 420 extends from the top surface of the body part 410, and can be spaced apart from the upper end of the suction part 300 by a predetermined distance when it is fastened to the suction part 300.

The head part 420 may include a first packing 421 and a second packing 425.

The first packing 421 extends from the upper end of the body part 410 and may have an annular shape.

The second packing 425 is disposed at the upper end of the first packing 421, is attachable to and detachable from the first packing 421, and may have an annular shape.

The second packing 425 may include a coupling projection 426 and an elastic part 427.

The coupling projection 426 may protrude from the bottom surface of the second packing 425 such that as the contact surface area between the first packing 421 and the second packing 425 primarily increases, the fastening force of the first packing 421 and the second packing 425 can be improved.

A filter (not shown) may be coupled to the bottom of the coupling projection 426. The shape of the filter may be a lattice shape and may have other well-known shapes.

Large-volume secretions, other than skin impurities, are allowed to enter the housing by the suction part 300. Such large-volume secretions may cause malfunction and damage to the absorber module. The absorber module according to an embodiment of the present disclosure can prevent malfunction and damage to the device by preventing ingress of external substances, other than skin impurities, because a filter is disposed between the first packing 421 and the second packing 425.

Further, the filter part 430 is attachable to and detachable from the second packing 425, so there is provided an advantage in hygiene management by cleaning or replacing the filter part 430 with a new filter.

The elastic part 427 may be disposed between the second packing 425 and the coupling projection 426 to be able to prevent damage to the second packing 425 due to impact that is generated during attachment and detachment.

The elastic part 427 may include a protrusion 428.

The protrusion 428 may protrude from the bottom surface of the elastic part 427 such that as the contact surface area between the first packing 421 and the second packing 425 secondarily increases, the fastening force of the first packing 421 and the second packing 425 can be improved.

The protrusion 428 may have a predetermined slope so that as the contact surface area between the first packing 421 and the second packing 425 tertiarily increases, the fastening force of the first packing 421 and the second packing 425 can be improved.

The first packing 421 may include an accommodation groove 422.

The accommodation groove 422 may be disposed on the top surface of the first packing 421 to be able to accommodate the protrusion 428.

The accommodation groove 422 may have an inclination corresponding to the slope of the protrusion 428 so that the protrusion 428 can be inserted therein.

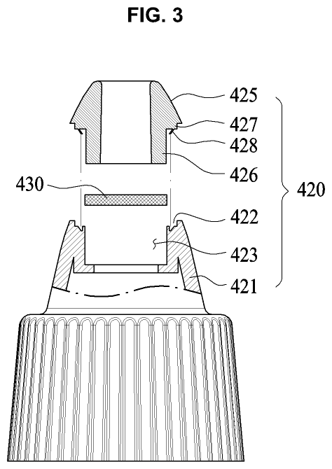

FIG. 3 is a perspective view of the adjustment part 210 according to an embodiment of the present disclosure.

The adjustment part 210 may include a button part 220 and a panel part 230.

The button part 220 allows a user to adjust the suction intensity of the suction part 300 stepwise and selectively through pressing operations.

The panel part 230 may be disposed on the bottom surface of the button part 220 to be able to support the button part 220.

A user can control the suction time, suction intensity, etc. of the absorber module by operating the button part 220.

In more detail, it is possible to sequentially increase the suction time such as 1 second, 2 seconds, 3 seconds, 4 seconds, 5 seconds, 10 seconds, 15 seconds, 20 seconds, 30 seconds, 45 seconds, or 60 seconds. Further, the absorber module can be repeatedly operated in a single cycle consisting of a suction time and a standby time. The cycle may be composed of a suction time, which is selected from at least one of 1 second, 2 seconds, 3 seconds, 4 seconds, 5 seconds, 10 seconds, 15 seconds, 20 seconds, 30 seconds, 45 seconds, or 60 seconds, and a standby period, which is selected from at least one of 1 second, 2 seconds, 3 seconds, 4 seconds, 5 seconds, 10 seconds, 15 seconds, 20 seconds, 30 seconds, 45 seconds, or 60 seconds. The cycle may be repeatedly performed two or more times (1 to 10 times or more).

Further, a user can select any one suction intensity of at least one among 40, 50, 60, 65, 70, 75, 80, 85, 90, 95, or 100 kPa by pressing the button part 220.

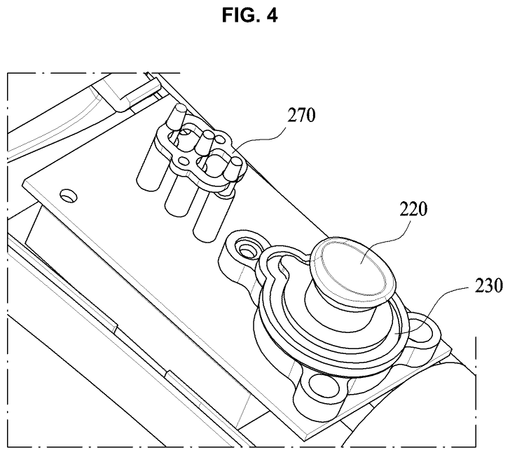

FIG. 4 is a detailed view of the button part 220 according to an embodiment of the present disclosure.

The button part 220 may include a first eccentric portion 221 and a second eccentric portion 222.

The first eccentric portion 221 may be formed in a shape in which the height of the button part 220 gradually increases toward its outer surface from the center of a button for the user's operational convenience, as shown in FIG. 4.

The second eccentric portion 222 may have a shape in which the height of the button part 220 gradually decreases toward its outer surface from the outer surface of the first eccentric portion 221, as shown in FIG. 4, to guide the user to press the first eccentric portion 221.

As shown in FIG. 4, the first eccentric portion 221 and the second eccentric portion 222 may be an elliptical shape, but unlike this, may have a circular shape and may have other well-known shape such as a triangle and a rectangle.

The side of the second eccentric portion 222, as shown in FIG. 4, may have a shape in which its width gradually narrows downward from the top surface of the button part 220 along the longitudinal axis of the button part 220.

As a result, it is possible to prevent damage to the button part 220 and the housing 200 due to friction when the button part 220 is pressed by a user.

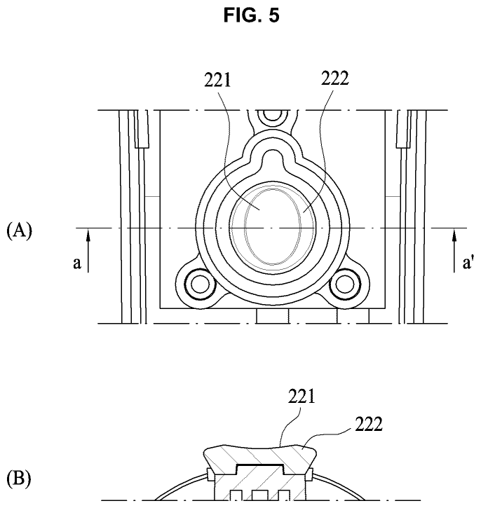

FIG. 5 is an exploded view of the panel part 230 according to an embodiment of the present disclosure.

The panel part 230 may include a first panel 240, a second panel 250, and a third panel 260.

The first panel 240 may be disposed on the bottom surface of the button part 220 to be able to support the button part 220 and primarily prevent external substances from entering the housing 200.

The second panel 250 may be disposed on the bottom surface of the first panel 240 to be able to secondarily prevent external substances from entering the housing 200.

The third panel 260 may be disposed on the bottom surface of the second panel 250 to be able to support the first panel 240 and the second panel 250.

As shown in FIG. 5(A), the first panel 240, the second panel 250, and the third panel 260 may be formed in a circular shape, but, unlike this, may have an elliptical shape and may have other well-known shapes such as a triangle and a rectangle.

The first panel 240 may include a first transfer portion 241 and the second panel 250 may include a second transfer portion 251.

The first transfer portion 241 protrudes from the bottom surface of the first panel 240, so it can primarily transfer pressure applied downward to the button part 220 by a user to an adjustment module 270.

The second transfer portion 251, as shown in FIG. 5(B), is engraved on the bottom surface of the second panel 250, so it can secondarily transfer pressure applied downward to the first transfer portion 241 to the adjustment module 270, but, unlike this, it may be embossed from the top surface of the second panel 250.

As a result, when a user presses the button part 220, the pressure is sequentially applied from the button part 220 to the first panel 240, from the first panel 240 to the second panel 250, and from the second panel 250 to the third panel 260, so a user can feel a smooth and sequential operation feeling and the durability of the absorber module may be improved by preventing malfunction of the button part 220.

The first panel 240 may include a guider 242 and the second panel 250 may include a guider accommodation portion 252.

The guider 242 may protrude from the side of the first panel 240 such that the first panel 240 and the second panel 250 can be fastened in a predetermined coupling direction. The guider accommodation portion 252 may further protrude from the second panel 250 to be able to accommodate the guider 242.

As a result, when the first panel 240 and the second panel 250 are fastened in a direction in which the guider 242 is not inserted into the guider accommodation portion 252, the first panel 240 and the second panel 250 are prevented from being coupled, so it is possible to prevent malfunction and damage to the device. When the first panel 240 and the second panel 250 are fastened in a direction in which the guider 242 is inserted into the guider accommodation portion 252, the guider 242 and the guider accommodation portion 252 are not misaligned with each other, so first panel 240 and the second panel 250 can be fastened to each other.

The third panel 260 may further include a coupling groove 261.

The coupling groove 261 protrudes from the side of the third panel 260 to be fixedly coupled to the adjustment module 270 and may include an accommodation space in which a coupling member, etc. can be inserted, and a plurality of coupling grooves may be formed.

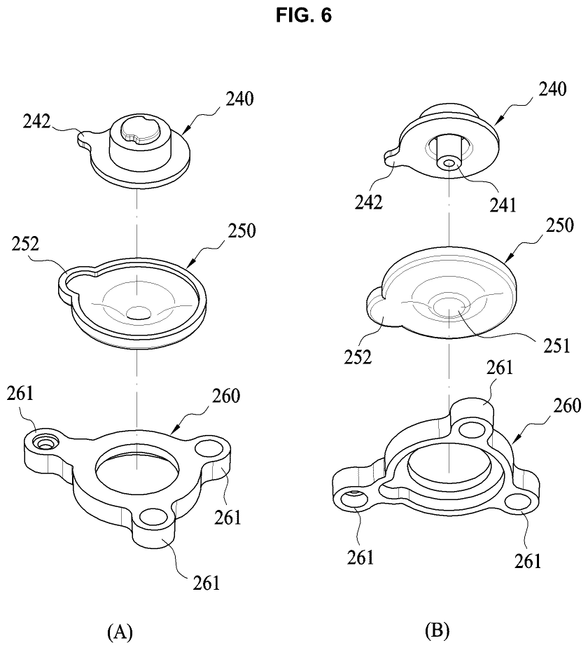

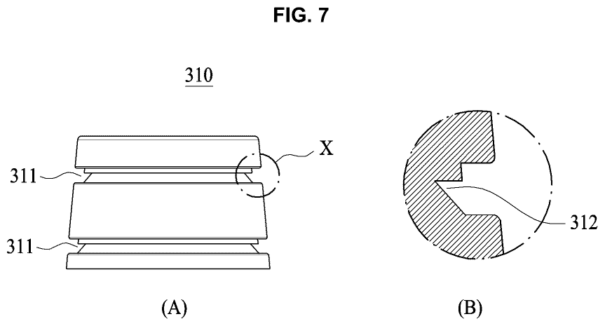

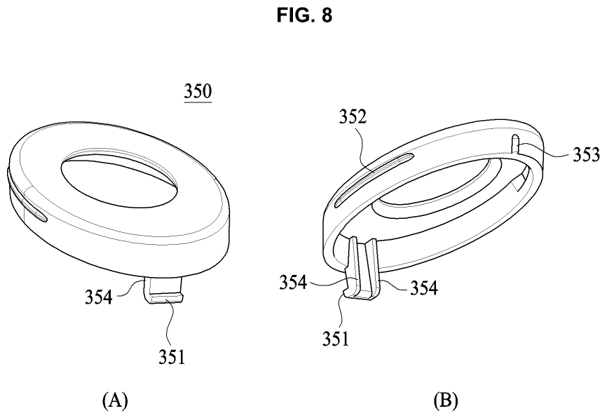

FIG. 6 and FIG. 7 are detailed views of a suction part according to an embodiment of the present disclosure.

The suction part 300 may include an upper suction part 310 and a lower suction part 350.

The upper suction part 310 may include a first sliding groove 311 and a second sliding groove 312.

The first sliding groove 311 and the second sliding groove 312 can accommodate an adjustment projection (not shown) formed on the inner circumferential surface of a body part 410 of the cap 400.

As a result, the adjustment projection of the body part 410, as shown in FIG. 6(B), is inserted in the first sliding groove 311 and sequentially inserted in the second sliding groove 312, so it is possible to adjust the inclination of the cap 400. The cap 400 of the absorber module according to the present disclosure can be separated from the suction part 300, and it possible to remove foreign substances from the skin surface by inserting the cap 400 into the first sliding groove 311 or the second sliding groove 312, depending on the user's needs.

The lower suction part 350 may include a locking portion 351, a stopper 352, an assistant locking portion 353, and a guide lane 354.

The locking portion 351 protrudes from the bottom surface of the lower suction part 350 and can be fixed to a side of the housing 100.

The stopper 352 may protrude from the side of the lower suction part 350 to be able to prevent idling of the suction part 300 after the suction part 300 is fastened to the housing 100. Further, an end of the stopper 352 is cut at an angle, so it is possible to prevent wear due to friction.

The assistant locking portion 353 may be disposed opposite the locking portion 351.

The guide lane 354, as shown in FIG. 7(A), may protrude from the outer surface of the locking portion 351, and, as shown in FIG. 7(B), may be engraved on the inner surface of the locking portion 351. As a result, the direction in which the suction part 300 is fastened to the housing 100 is guided, so that a user can manage the absorber module by more easily attaching to or separating from the suction part 300 from the housing 100.

The embodiments described herein are merely illustrative the technical spirit of the present disclosure, and may be changed and modified in various ways without departing from the essential characteristics of the present disclosure by those skilled in the art.

Accordingly, the embodiments are provided merely not to limit, but to explain the technical spirit of the present disclosure, and the spirit of the present disclosure is not limited by the embodiments.

The protection range of the present disclosure should be construed on the basis of claims and all technical spirits that are considered as being same as or equivalent to the protection range should be construed as being included in the right range of the present disclosure.
10—suction module
11—dome-shaped protrusion
100—housing
200—housing
210—adjustment part
220—button part
221—first eccentric portion
222—second eccentric portion
230—panel part
240—first panel
241—first transfer portion
242—guider
250—second panel
251—second transfer portion
252—guider accommodation portion
260—third panel
261—coupling groove
270—adjustment module
300—suction part
310—upper suction part
311—first sliding groove
312—second sliding groove
350—lower suction part
351—locking portion
352—stopper
353—assistant locking portion 353,
354—guide lane
400—cap
410—body part
420—head part
421—first packing
422—accommodation groove
423—accommodation space
425—second packing
426—coupling projection
427—elastic part
428—protrusion
430—filter part

1. An absorber module to prevent contamination due to foreign substances and secretions, comprising:
a housing having a suction module therein and including an adjustment part, which can adjust suction intensity, on an outer surface thereof;
a suction part connected to an upper end of a body part, being able to generate negative pressure outside, and having a flat top surface; and
a cap blocking ingress of external substances by covering an outer surface of the suction part to be able to prevent the suction part from being contaminated by external substances, and being attachable to and detachable from an upper end of the suction part,
wherein the cap includes:
a body part detachably coupled to a side of the suction part; and
a head part extending from a top surface of the body part and spaced apart from the upper end of the suction part by a predetermined distance when fastened to the suction part.

2. The absorber module of claim 1, wherein the head part includes:
an annular first packing extending from the upper end of the body part;
an annular second packing disposed at an upper end of the first packing and being attachable to and detachable from the first packing; and
a filter part disposed between the first packing and the second packing to maintain hygiene and cleanliness, and being replaceable.

3. The absorber module of claim 2, wherein the second packing includes a coupling projection protruding from a bottom surface of the second packing so that as a contact surface area between the first packing and the second packing primarily increases, a fastening force of the first packing and the second packing can be improved.

4. The absorber module of claim 3, wherein the second packing includes an elastic part disposed between the second packing and the coupling projection to be able to prevent damage to the second packing due to impact that is generated during attachment and detachment.

5. The absorber module of claim 4, wherein the elastic part includes a protrusion protruding from a bottom surface of the elastic part so that as the contact surface area between the first packing and the second packing secondarily increases, the fastening force of the first packing and the second packing can be improved, and
the first packing includes an accommodation groove disposed on a top surface thereof to be able to accommodate the protrusion.

6. The absorber module of claim 5, wherein the protrusion has a predetermined slope so that as the contact surface area between the first packing and the second packing tertiarily increases, the fastening force of the first packing and the second packing can be improved, and
the accommodation groove has an inclination corresponding to the slope of the protrusion so that the protrusion can be inserted therein.
